(19) United States
(12) Patent Application Publication
(10) Pub. No.: US 2023/0397859 A1
(43) Pub. Date: Dec. 14, 2023

(54) BLOOD COLLECTION DEVICES, SYSTEMS, AND METHODS

(71) Applicant: Becton, Dickinson and Company, Franklin Lakes, NJ (US)

(72) Inventors: Bo Yan, Shanghai (CN); Xiwei Chen, Shanghai (CN); Simon Liu, Shanghai (CN); Yueqiang Xue, Shanghai (CN); Yijie Wu, Shanghai (CN); Jithendra Kumar, Singapore (SG); Tao Jiang, Shanghai (CN)

(21) Appl. No.: 18/033,771

(22) PCT Filed: Nov. 1, 2021
(86) PCT No.: PCT/US2021/057501

§ 371 Date: Apr. 25, 2023

(30) Foreign Application Priority Data
Nov. 6, 2020 (CN) 202011226876.X

(51) Int. Cl. A61B 5/15; A61B 5/153; A61M 39/10; A61M 39/24

(52) U.S. Cl. CPC A61B 5/150992; A61B 5/153; A61B 5/150221; A61M 39/10; A61M 39/24

(57) ABSTRACT

A blood draw system may include a catheter adapter connector in fluid communication with a catheter adapter, and a blood draw device in selective fluid communication with the catheter adapter connector. A first valve may be utilized to selectively permit fluid communication between the blood draw device and the catheter adapter connector. A flush device may also be in selective fluid communication with the catheter adapter connector to flush the catheter adapter after a blood draw procedure. A second valve may be utilized to selectively permit fluid communication between the flush device and the catheter adapter connector.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter is an over-the-needle peripheral intravenous catheter (“PIVC”). As its name implies, the over-the-needle PIVC may be mounted over an introducer needle having a sharp distal tip. The PIVC and the introducer needle may be assembled so that the distal tip of the introducer needle extends beyond the distal tip of the PIVC with the bevel of the needle facing away from skin of the patient. The PIVC and the introducer needle are typically inserted at a shallow angle through the skin and into a blood vessel of the patient, such as an artery, a vein, or any other vasculature of the patient. Once the PIVC has been properly placed within the blood vessel, the introducer needle may be withdrawn and the PIVC may be secured within the blood vessel by securing a catheter adapter (coupled with the PIVC) to the skin of the patient with dressing. Other common types of catheters include, but are not limited to, peripherally inserted central catheters (“PICC”) and central venous catheters (“CVC”).

A properly placed catheter may be utilized by a clinician to withdraw blood from the patient. Moreover, blood draw procedures are typically only performed with a newly inserted catheter in order to avoid certain risks. For example, during a blood draw procedure the clinician will typically: (1) insert a new catheter/catheter adapter; (2) couple a blood draw device to the catheter adapter; (3) collect a blood sample from the patient with the blood draw device; (4) decouple the blood draw device from the catheter adapter; (4) couple a flush device to the catheter adapter; (5) flush the catheter/catheter adapter with the flush device; and (6) decouple the flush device from the catheter adapter.

However, each of these steps introduce complexity and inefficiencies to the blood draw procedure (e.g., multiple connection/disconnection steps), limit blood draw procedures to newly inserted catheters, and increase certain risks to the patient. Examples of risks that may be increased include drug contamination of the blood sample, hemolysis of the blood sample, increased risk of infection, increased risk of catheter occlusion, increased risk of catheter migration, etc.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure generally relates to blood collection devices, systems, and methods. The various blood collection devices, systems, and methods of the present disclosure have been developed in response to the present state of the art, and in particular, in response to the problems and needs in the art that have not yet been fully solved by currently available devices, systems, and methods for collecting blood from a patient.

In some embodiments, a blood draw system may include at least one catheter adapter connector configured to engage a catheter adapter and place the at least one catheter adapter connector in fluid communication with the catheter adapter. The blood draw system may also include at least one blood draw device which may be in selective fluid communication with the at least one catheter adapter connector. The at least one blood draw device may be configured to draw blood from a blood vessel of a patient. A first valve may be configured to selectively permit fluid communication between the at least one blood draw device and the at least one catheter adapter connector. A flush device may be in selective fluid communication with the at least one catheter adapter connector and the flush device may be configured to flush the catheter adapter with fluid ejected from the flush device. A second valve may be configured to selectively permit fluid communication between the flush device and the at least one catheter adapter connector.

In some embodiments, the at least one catheter adapter connector may include at least one of a needle, a wing needle set, a needleless connector, a luer lock connector, a lure slip connector, a luer taper connector, an extension set, and an extension tube.

In some embodiments, the at least one blood draw device may include at least one of a syringe and a vacutainer.

In some embodiments, the first valve and the second valve may include at least one of a stopcock valve, a two-way stopcock valve, a three-way stopcock valve, a check valve, a slide clamp, and a pinch clamp.

In some embodiments, the flush device may include a syringe filled with a saline solution.

In some embodiments, the blood draw system may further include at least one fluid conduit coupled to the at least one catheter adapter connector. The at least one fluid conduit may be configured to place at least one of the flush device and the at least one blood draw device in fluid communication with the at least one catheter adapter connector.

In some embodiments, the at least one catheter adapter connector may include a needle that is at least partially housed within a protective needle housing.

In some embodiments, a protective needle housing system may include a needle and a protective needle housing. The protective needle housing may include a needle passageway configured to receive at least a portion of the needle therein and a needle block configured to move between a closed position and an open position. In the closed position, the needle block may prevent the needle from advancing distally through the needle passageway. In the open position, an aperture formed in the needle block may be placed in alignment with the needle passageway to allow the needle to advance distally through the needle passageway.

In some embodiments, the protective needle housing system may also include a resilient member configured to bias the needle block in the closed position.

In some embodiments, the resilient member include at least one of a spring clip and a helical spring.

In some embodiments, the needle block may include a first engagement surface configured to engage with a second engagement surface of a catheter adapter, such that, as the second engagement surface of the catheter adapter engages the first engagement surface of the needle block, the needle block is moved from the closed position to the open position.

In some embodiments, the protective needle housing may include an engagement feature configured to couple the protective needle housing to a catheter adapter.

In some embodiments, a distal end of the needle may include a closed tip and an aspiration aperture formed in a sidewall of the needle proximate the closed tip.

In some embodiments, the needle may include a bumper configured to prevent the needle from moving proximally out of the protective needle housing.

In some embodiments, a method of drawing blood from a patient via a blood draw system may include coupling at least one catheter adapter connector to a catheter adapter in order to place the at least one catheter adapter connector in fluid communication with the catheter adapter. The method may also include adjusting a first valve to permit fluid communication between at least one blood draw device and the at least one catheter adapter connector. The at least one blood draw device may be configured to draw blood from a blood vessel of a patient. The method may also include aspirating blood into the at least one blood draw device through the at least one catheter adapter connector. The method may also include adjusting the first valve to prevent fluid communication between the at least one blood draw device and the at least one catheter adapter connector. The method may further include adjusting a second valve to permit fluid communication between a flush device and the at least one catheter adapter connector. The method may additionally include flushing the catheter adapter with fluid ejected from the flush device.

In some embodiments, the method may also include adjusting the second valve to prevent fluid communication between the flush device and the at least one catheter adapter connector and to permit fluid communication between a second blood draw device and the at least one catheter adapter connector. The method may also include aspirating blood into the second blood draw device through the at least one catheter adapter connector.

In some embodiments, the at least one catheter adapter connector may include a first catheter adapter connector and a second catheter adapter connector.

In some embodiments, the at least one catheter adapter connector may include at least one of a needle, a wing needle set, a needleless connector, a luer lock connector, a lure slip connector, a luer taper connector, an extension set, and an extension tube.

In some embodiments, the at least one catheter adapter connector may include a needle that is at least partially housed within a protective needle housing.

In some embodiments, the first valve and the second valve may include at least one of a stopcock valve, a two-way stopcock valve, a three-way stopcock valve, a check valve, a slide clamp, and a pinch clamp.

It is to be understood that both the foregoing general description and the following detailed description are examples and explanatory and are not restrictive of the embodiments of the present disclosure, as claimed. It should be understood that the various embodiments of the present disclosure are not limited to the arrangements and instrumentality shown in the drawings. It should also be understood that the embodiments of the present disclosure may be combined, or that other embodiments may be utilized and that structural changes, unless so claimed, may be made without departing from the scope of the various embodiments of the present disclosure. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is a perspective top view of an example catheter system, according to some embodiments;

FIG. 1B is a cross-sectional top view of the catheter system of FIG. 1A, according to some embodiments;

FIG. 2A is a perspective view of a protective needle housing, according to some embodiments;

FIG. 2B is a front view of the protective needle housing of FIG. 2A in a closed position, according to some embodiments;

FIG. 2C is a front view of the protective needle housing of FIG. 2A in an open position, according to some embodiments;

FIG. 2D is a cross-sectional side view of the protective needle housing of FIG. 2A in a closed position, according to some embodiments;

FIG. 2E is a cross-sectional side view of the protective needle housing of FIG. 2A in an open position, according to some embodiments;

FIG. 3A is a side view of a needle, according to some embodiments;

FIG. 3B is a close up view of a distal end of the needle of FIG. 3A, according to some embodiments;

FIG. 3C is a close up view of an intermediate portion of the needle of FIG. 3A, according to some embodiments;

FIG. 4A is a cross-sectional side view of a protective needle housing system in a closed position, according to some embodiments;

FIG. 4B is a cross-sectional side view of the protective needle housing system of FIG. 4A in an open position, according to some embodiments;

FIG. 5 is a cross-sectional side view of an example catheter adapter, according to some embodiments;

FIG. 6 is a cross-sectional side view of the catheter adapter of FIG. 5 coupling to the protective needle housing system of FIG. 4A in a closed position, according to some embodiments;

FIG. 7 is a cross-sectional side view of the catheter adapter of FIG. 5 coupled to the protective needle housing system of FIG. 4A in an open position, according to some embodiments;

FIG. 8 is a cross-sectional side view of the catheter adapter of FIG. 5 coupled to the protective needle housing system of FIG. 4A in an open position with the needle moved distally within the catheter adapter, according to some embodiments;

FIG. 9A is a front view of a protective needle housing in a closed position, according to an alternative embodiment;

FIG. 9B is a front view of the protective needle housing of FIG. 9A in an open position, according to an alternative embodiment;

FIG. 10 is a side view of a blood collection set, according to some embodiments;

FIG. 11 is a side view of an alternative blood collection set, according to some embodiments;

FIG. 12 is a side view of an alternative blood collection set, according to some embodiments;

FIG. 13 is a side view of an alternative blood collection set, according to some embodiments;

FIG. 14 is a side view of an alternative blood collection set, according to some embodiments;

FIG. 15 is a side view of an alternative blood collection set, according to some embodiments;

FIG. 16 is a side view of an alternative blood collection set, according to some embodiments; and

FIG. 17 is a flowchart of a method for drawing blood from a patient via a blood draw system, according to some embodiments.

It is to be understood that the Figures are for purposes of illustrating the concepts of the present disclosure and may not be drawn to scale. Furthermore, the Figures illustrate example embodiments and do not represent limitations to the scope of the present disclosure.

### DESCRIPTION OF EMBODIMENTS

Example embodiments of the present disclosure may be best understood by reference to the Figures, wherein like parts are designated by like numerals throughout. It will be readily understood that the components of the present disclosure, as generally described and illustrated in the Figures herein, could be arranged and designed in a wide variety of different configurations. Thus, the following more detailed description of the embodiments of the apparatus and systems, as represented in the Figures, is not intended to limit the scope of the present disclosure, as claimed in this or any other application claiming priority to this application, but is merely representative of example embodiments of the present disclosure.

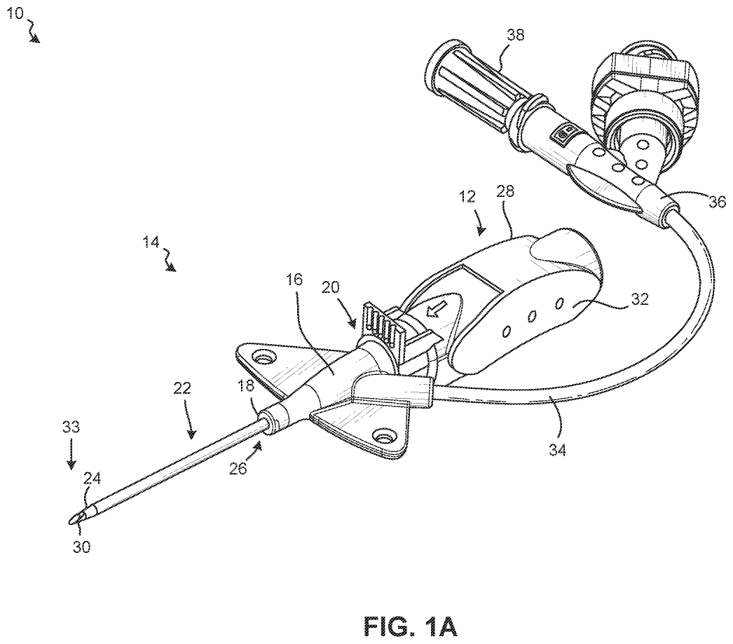

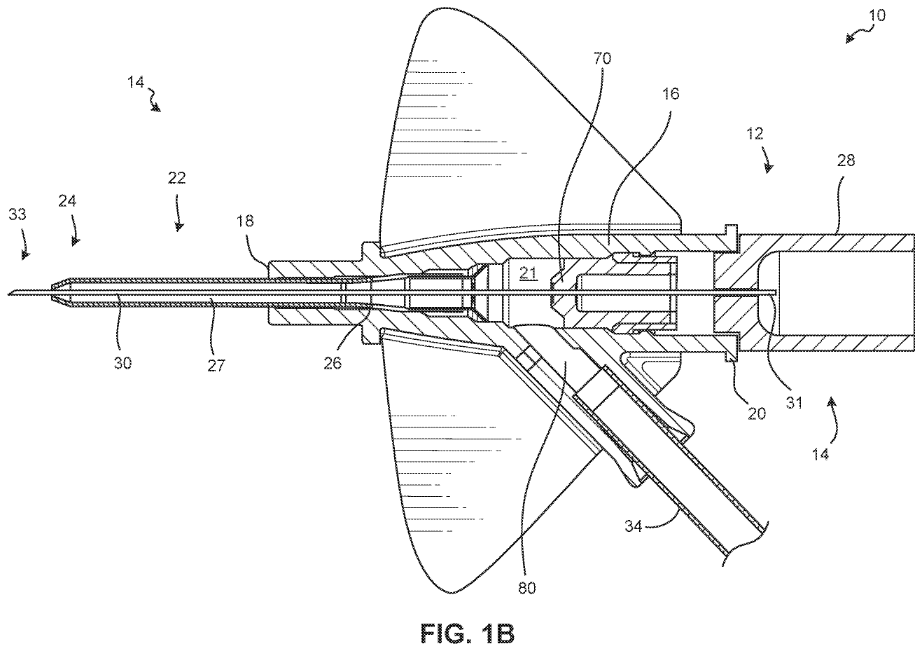

Referring to FIGS. 1A and 1, in some embodiments, a catheter system 10 may include a needle assembly 12 and a catheter assembly 14, according to some embodiments. FIGS. 1A and 1B illustrate the catheter system 10 in an insertion position, ready for insertion into a vein of a patient (not shown). In some embodiments, the catheter assembly 14 may include a catheter adapter or catheter adapter body 16, which may include a proximal end 20, a distal end 18, and a catheter adapter channel 21 formed within the catheter adapter body 16 and extending between the proximal and distal ends 20, 18 of the catheter adapter body 16. In some embodiments, the catheter adapter body 16 may include a septum 70 coupled to the catheter adapter body 16 adjacent the catheter adapter channel 21. In some embodiments, the septum 70 may be a single component septum. In some embodiments, the septum 70 may be a multi-component septum. In some embodiments, the catheter assembly 14 may include a catheter 22, which may include a proximal end 26, a distal end 24, and a catheter lumen 27 extending between the proximal and distal ends 26, 24 of the catheter 22. In some embodiments, the catheter 22 may include a peripheral intravenous catheter (“PIVC”). In some embodiments, the proximal end 26 of the catheter 22 may be secured within the catheter adapter body 16.

In some embodiments, the needle assembly 12 may include a needle hub 28, which may be removably coupled to the catheter adapter body 16. In some embodiments, the needle assembly 12 may include an introducer needle 30. In some embodiments, a proximal end 31 of the introducer needle 30 may be secured within the needle hub 28. In some embodiments, the introducer needle 30 may extend through the catheter lumen 27 and a distal end 33 of the introducer needle 30 may protrude from the distal end 24 of the catheter 22 when the catheter system 10 is in an insertion position and ready for insertion into a vein of a patient.

In some embodiments, the needle assembly 12 may include a needle grip 32, which a clinician may grip and move proximally to withdraw the introducer needle 30 from the vein once placement of the catheter 22 within the vein is confirmed. In some embodiments, the catheter system 10 may include an extension tube 34. In some embodiments, a distal end of the extension tube 34 may be coupled to the catheter adapter body 16 and a proximal end of the extension tube 34 may be coupled to an adapter 36. In some embodiments, the catheter adapter body 16 may include an access port 80, which may be in fluid communication with the catheter adapter channel 21. In some embodiments, a distal end of the extension tube 34 may be coupled to the access port 80, such that the extension tube 34 may be in fluid communication with the catheter adapter channel 21 via the access port 80.

In some embodiments, a fluid infusion device (not shown) may be coupled to the adapter 36 to deliver fluid to the patient via the catheter 22 inserted in the vein, once the introducer needle 30 is removed from the catheter system 10. In some embodiments, a blood collection device may be coupled to the adapter 36 to withdraw blood from the patient via the catheter 22 inserted within the vein.

The catheter system 10 may include straight, ported, integrated, and conventional catheters. For example, in some embodiments, the catheter system 10 may be integrated, having the extension tube 34 integrated within the catheter adapter body 16, such as, for example, the BD NEXIVA™ Closed IV Catheter System, the BD NEXIVA™ DIFFUSICS™ Closed IV Catheter System, the BD PEGASUS™ Safety Closed IV Catheter System, or another integrated catheter system. In some embodiments, the catheter system 10 may be non-integrated, without the extension tube 34.

In some embodiments, the catheter system 10 may be vented to observe blood and facilitate proximal flow of blood within the introducer needle 30 and/or the catheter 22. In some embodiments, the catheter system 10 may be vented in any suitable manner. For example, a vent plug 38 may be coupled to the adapter 36 during insertion of the catheter 22 into the patient. In some embodiments, the vent plug 38 may be permeable to air but not to blood. In some embodiments, the catheter 22, the catheter adapter body 16, the extension tube 34, the adapter 36, and the vent plug 38 may be in fluid communication. As another example, in some embodiments, the needle hub 28 may include a flash chamber.

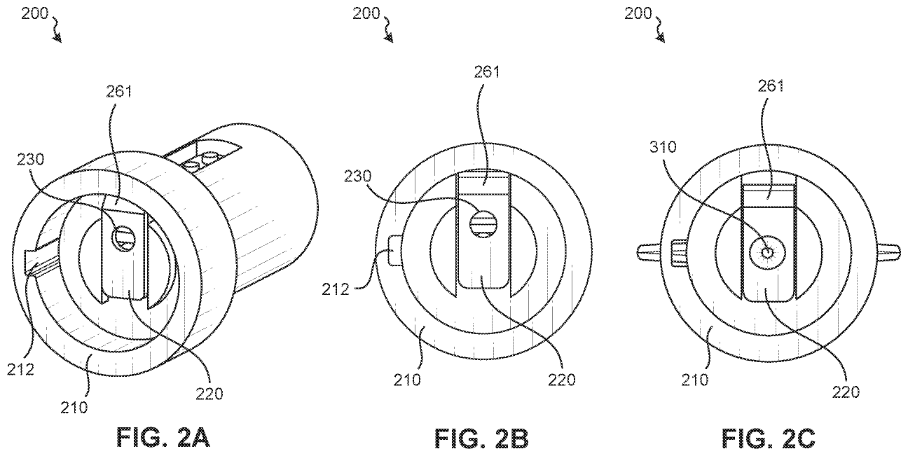

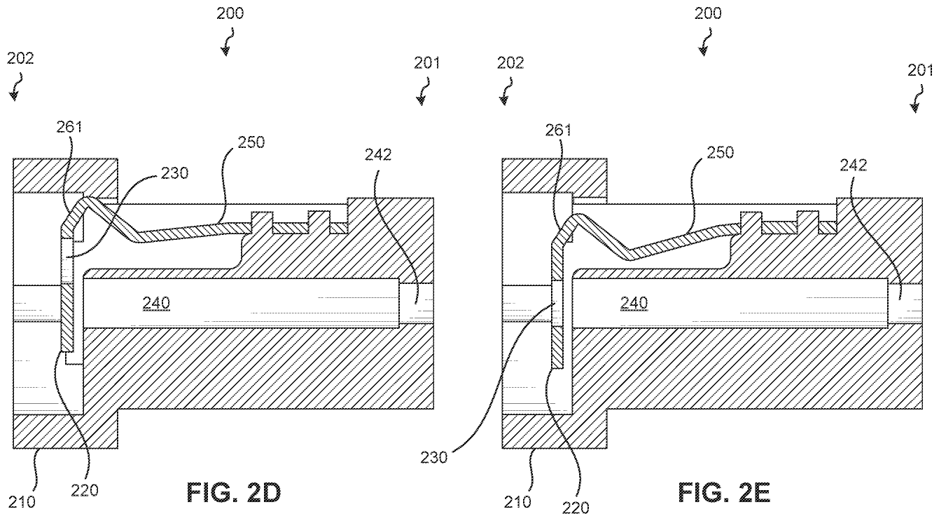

FIGS. 2A-2E illustrate various views of a protective needle housing 200, according to some embodiments. Specifically, FIG. 2A is a perspective view of the protective needle housing 200; FIG. 2B is a front view of the protective needle housing 200 in a closed position; FIG. 2C is a front view of the protective needle housing 200 in an open position; FIG. 2D is a cross-sectional side view of the protective needle housing 200 in a closed position; and FIG. 2E is a cross-sectional side view of the protective needle housing 200 in an open position.

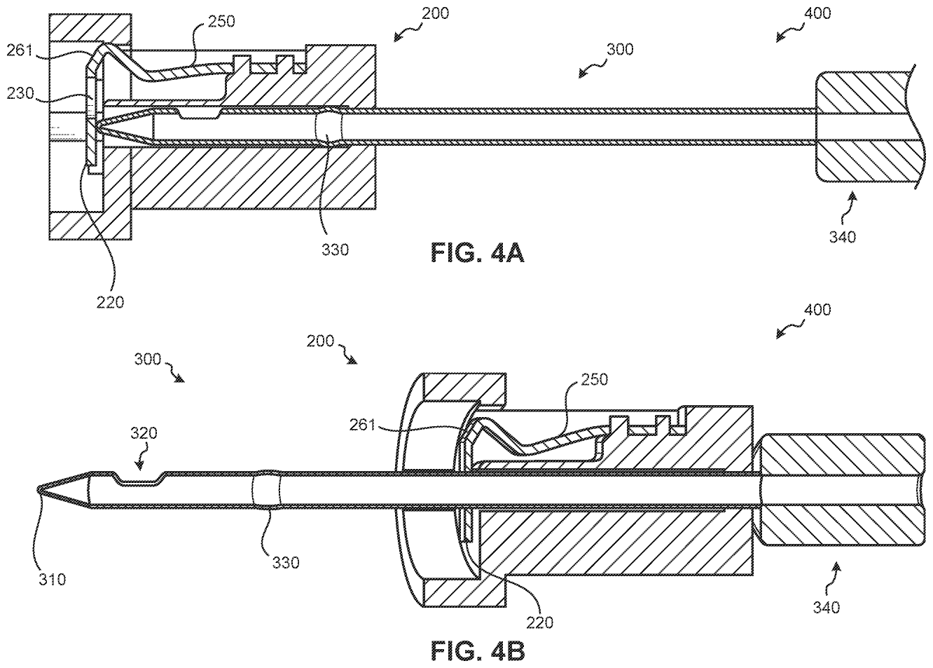

In general, the protective needle housing 200 may include a proximal end 201, a distal end 202, an engagement feature 210 located at the distal end 202 of the protective needle housing 200, an engagement slot 212, a needle passageway 240 extending between the proximal and distal ends 201, 202 of the protective needle housing 200, a reduced inner diameter portion 242 of the needle passageway 240 located at the proximal end 201 of the protective needle housing 200, and a needle block 220 including one or more of: an aperture 230, a first engagement surface 261, and a resilient member 250. Operation of the protective needle housing 200 will be discussed in more detail below with respect to FIGS. 4A-8.

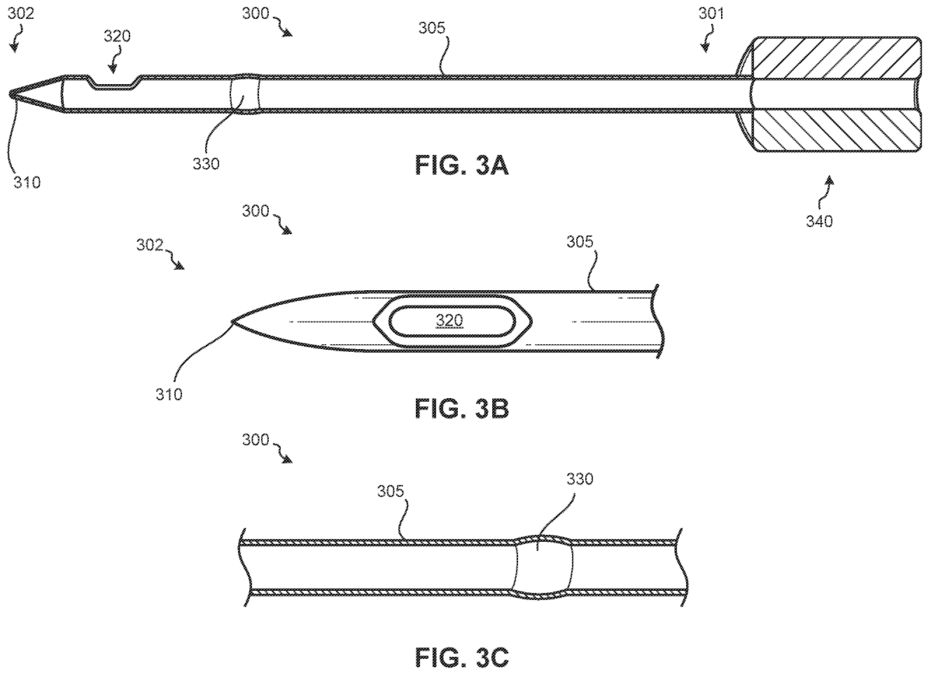

FIGS. 3A-3C illustrate various views of a needle 300, according to some embodiments. Specifically, FIG. 3A is a side view of the needle 300; FIG. 3B is a close up top view of a distal end of the needle 300; and FIG. 3C is a close up view of an intermediate portion of the needle 300.

In general, the needle 300 may include a needle cannula 305 extending between a proximal end 301 and a distal end 302 of the needle 300, a hub 340 coupled to the proximal end 301 of the needle 300, a closed tip 310 located at the distal end 302 of the needle 300, an aspiration aperture 320 formed in a sidewall of the needle cannula 305 located proximate the closed tip 310, and a bumper 330 coupled to the needle cannula 305. The operation of the needle 300 and the protective needle housing 200 will now be discussed with reference to FIGS. 4A-8.

FIGS. 4A and 4B illustrate various views of a protective needle housing system 400, according to some embodiments. Specifically, FIG. 4A is a cross-sectional side view of the protective needle housing system 400 in a closed position, and FIG. 4B is a cross-sectional side view of the protective needle housing system 400 in an open position. In general, the protective needle housing system 400 may include the needle 300 of FIGS. 3A-3C slidably coupled within the protective needle housing 200 of FIGS. 2A-2E.

The needle 300 may be held captive within the protective needle housing 200 in the proximal direction via the bumper 330 that protrudes from the needle cannula 305. The bumper 330 may have a larger outer diameter than the reduced inner diameter portion 242 of the needle passageway 240. Thus, the bumper 330 may be configured to prevent the needle 300 from moving too far in the proximal direction to retain the needle 300 within the protective needle housing 200 when a clinician pulls the needle 300 in the proximal direction. The needle 300 may also be held captive within the protective needle housing 200 in the distal direction via the needle block 220 when the needle block 220 is in the closed position (see FIG. 4A), or via the hub 340 when the needle block 220 is in the open position (see FIG. 4B). Thus, the needle block 220 and/or the hub 340 may prevent the needle 300 from moving too far in the distal direction to retain the needle 300 within the protective needle housing 200 when a clinician pushes the needle 300 in the distal direction. In this manner, the needle passageway 240 and/or the reduced inner diameter portion 242 of the needle passageway 240 may each be configured to receive at least a portion of the needle 300, and capturing the closed tip 310 of the needle 300 within the protective needle housing 200 provides an increased level of safety to the clinician against accidental needle punctures. The closed tip 310 may also reduce blood hemolysis and reduce tears and contamination caused by the closed tip 310 puncturing through a septum.

As previously noted, the needle block 220 may be configured to move between a closed position (e.g., see FIGS. 2A, 2B, 2D, and 4A) and an open position (e.g. see FIGS. 2C, 2E, and 4B). In the closed position, the needle block 220 may prevent the needle 300 from advancing distally through the needle passageway 240 by blocking the closed tip 310 of the needle 300 and preventing distal translation of the needle 300 through the needle passageway 240, as shown in FIG. 4A. In the open position, the aperture 230 formed in the needle block 220 may be placed in alignment with the needle passageway 240 to allow the needle 300 to advance distally through the needle passageway 240, as shown in FIG. 4B.

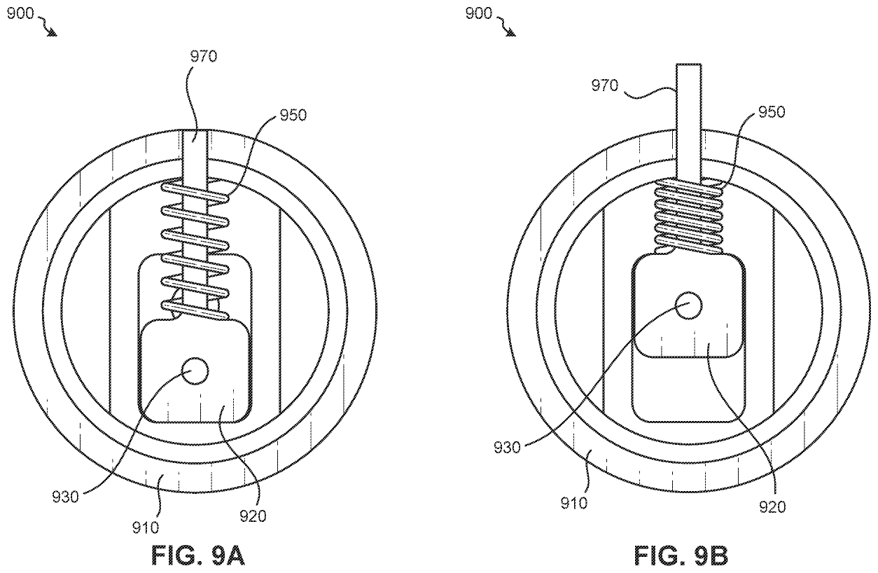

The resilient member 250 may be configured to apply a biasing force on the needle block 220 in order to preferentially bias the resilient member 250 in the closed position. In the embodiment shown if FIGS. 2A-2E, 4A, 4B, and 6-8, the resilient member 250 may include a spring clip configured to bias the needle block 220 in the upward/closed position. However, it will be understood that other biasing structures are contemplated herein (e.g., see FIGS. 9A and 9B).

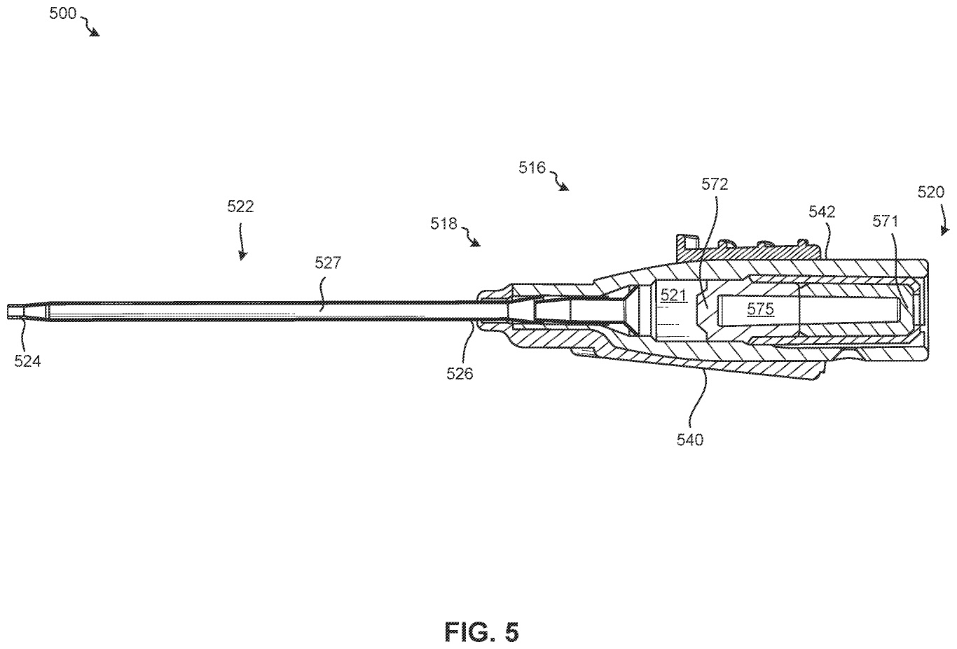

FIG. 5 illustrates a cross-sectional side view of a catheter adapter 500, according to some embodiments. In general, the catheter adapter 500 may include a catheter adapter body 516 and a catheter 522. The catheter adapter body 516 may include a proximal end 520, a distal end 518, an inferior surface 540, a superior surface 542, and a catheter adapter channel 521 formed within the catheter adapter body 516 and extending between the proximal and distal ends 520, 518 of the catheter adapter body 516. In some embodiments, the catheter adapter body 516 may include a first septum 571 and a second septum 572. The first septum 571 may be located toward the proximal end 520 of the catheter adapter body 516 and the second septum 572 may be coupled within the catheter adapter body 516 adjacent the catheter adapter channel 521. A septum channel 575 may be formed intermediate the first septum 571 and the second septum 572. In some embodiments, the septum 570 may be a multi-component septum. In some embodiments (not shown) the catheter adapter 500 septum may utilize a single component septum. In some embodiments, the catheter adapter body 516 may also include an access port (not shown in FIG. 5) that may be in fluid communication with the catheter adapter channel 521.

The catheter 522 may include a proximal end 526, a distal end 524, and a catheter lumen 527 extending between the proximal and distal ends 526, 524 of the catheter 522. In some embodiments, the catheter 522 may include a peripheral intravenous catheter (“PIVC”). In some embodiments, the proximal end 526 of the catheter 522 may be coupled to and/or secured within the catheter adapter body 516, such that the catheter lumen 527 may be in fluid communication with the catheter adapter channel 521.

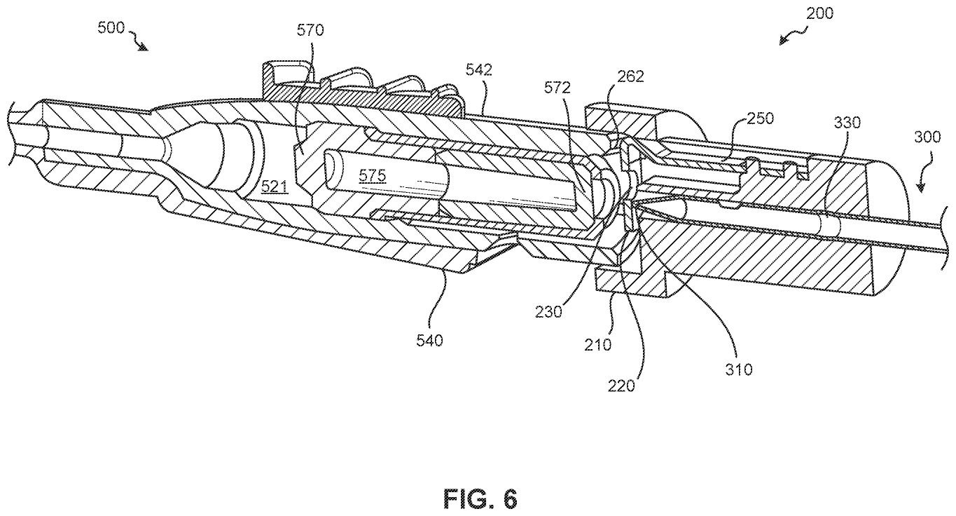

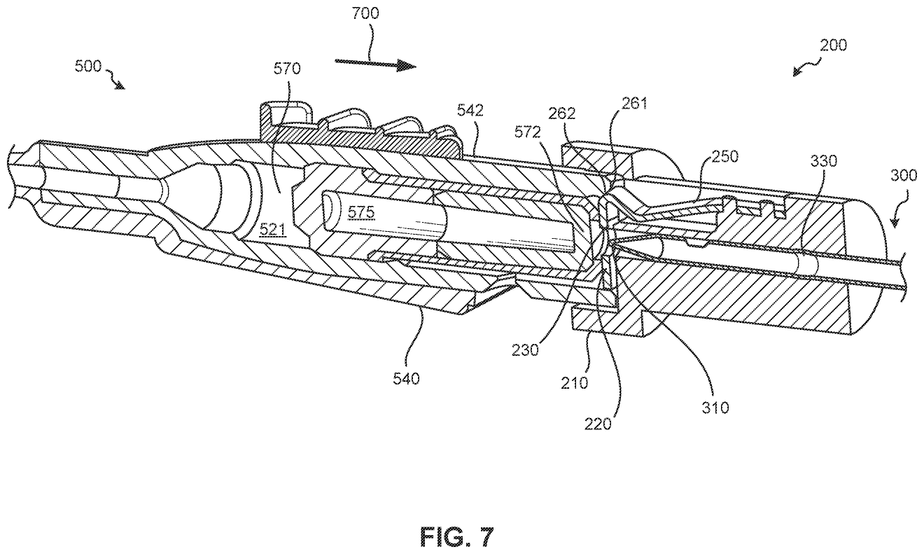

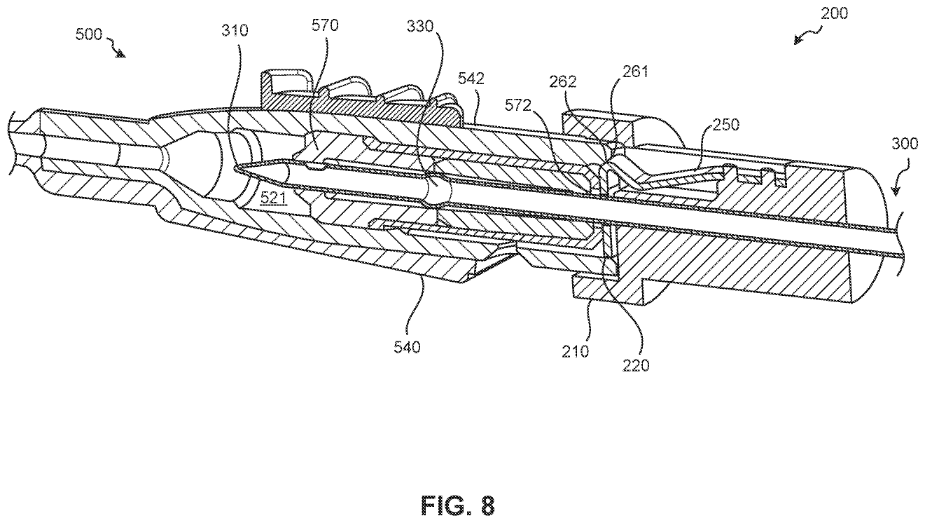

FIGS. 6-8 illustrate various views of the catheter adapter 500 of FIG. 5 coupling to the protective needle housing system 400 of FIG. 4A, according to some embodiments. Specifically, FIG. 6 is a cross-sectional side view of the catheter adapter 500 coupling to the protective needle housing system 400 in a closed position; FIG. 7 is a cross-sectional side view of the catheter adapter 500 fully coupled to the protective needle housing system 400 in an open position; and FIG. 8 is a cross-sectional side view of the catheter adapter 500 coupled to the protective needle housing system 400 in an open position with the needle 300 moved distally and projecting inside of the catheter adapter 500.

FIG. 6 illustrates the proximal end 520 of the catheter adapter 500 as it is slidably received within the engagement feature 210 of the protective needle housing 200. As the proximal end 520 of the catheter adapter 500 is received within the engagement feature 210 of the protective needle housing 200, the first engagement surface 261 of the needle block 220 will engage a second engagement surface 262 formed on the proximal end 520 of the catheter adapter 500. The first engagement surface 261 of the needle block 220 and the second engagement surface 262 of the catheter adapter 500 may each be angled toward the proximal direction. In this manner, the first engagement surface 261 may be configured to engage the second engagement surface 262 of the catheter adapter 500, such that, as the second engagement surface 262 of the catheter adapter 500 engages the first engagement surface 261 of the needle block 220, the needle block 220 moves from the closed position to the open position. Thus, when a clinician fully couples the protective needle housing 200 to the catheter adapter 500 by moving the catheter adapter 500 in the direction of arrow 700, the needle block 220 may automatically move from the closed position to the open position, as shown in FIG. 7. Once the needle block 220 is moved to the open position, the clinician is free to move the needle 300 distally to insert the needle 300 into the catheter adapter 500, as shown in FIG. 8.

FIGS. 9A and 9B illustrate two views of a protective needle housing 900, according to an alternative embodiment. Specifically, FIG. 9A is a front view of the protective needle housing 900 in a closed position and FIG. 9B is a front view of the protective needle housing 900 in an open position. In general, the protective needle housing 900 may include similar features and structures to the protective needle housing 200 shown in FIGS. 2A-2E, such as an engagement feature 910, a needle block 920 and an aperture 930. However, the protective needle housing 900 may utilize an alternative structure including a resilient member 950 (e.g., a helical spring) and needle block arm 970, which may be configured to toggle the needle block 920 between the open and closed positions. The needle block 920 may also be configured to automatically move to the open position upon coupling with the catheter adapter 500.

However, it will be understood that any number of different resilient member structures or configurations are envisioned herein which may be utilized to toggle a needle block between the open and closed positions and/or to apply a biasing force to the needle block toward the closed position. It will also be understood that any of the catheter adapter structures described herein and/or any of the protective needle housing structures or systems disclosed herein may be utilized with any of the blood draw collection sets/systems described below.

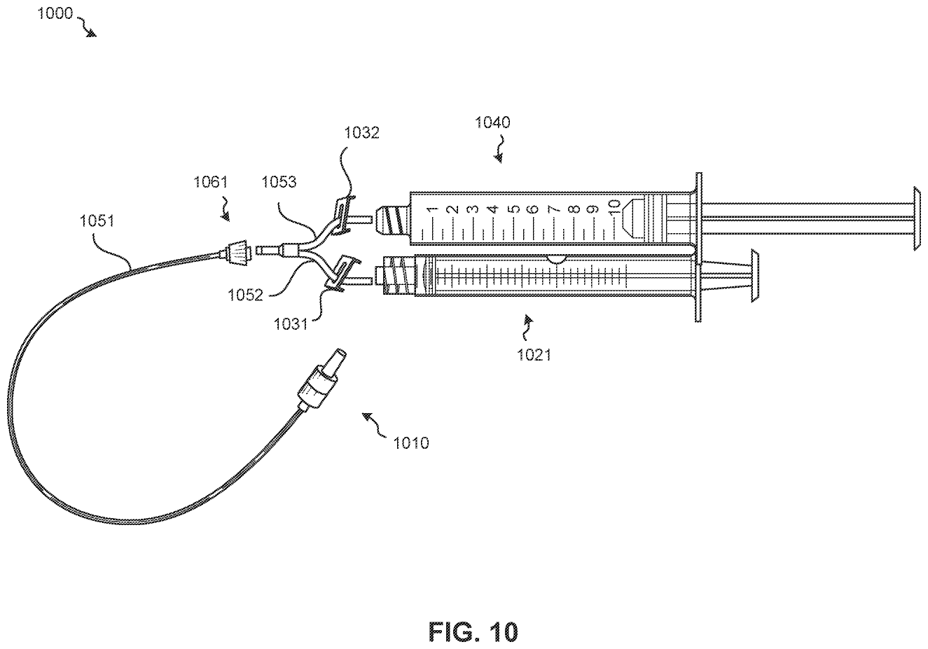

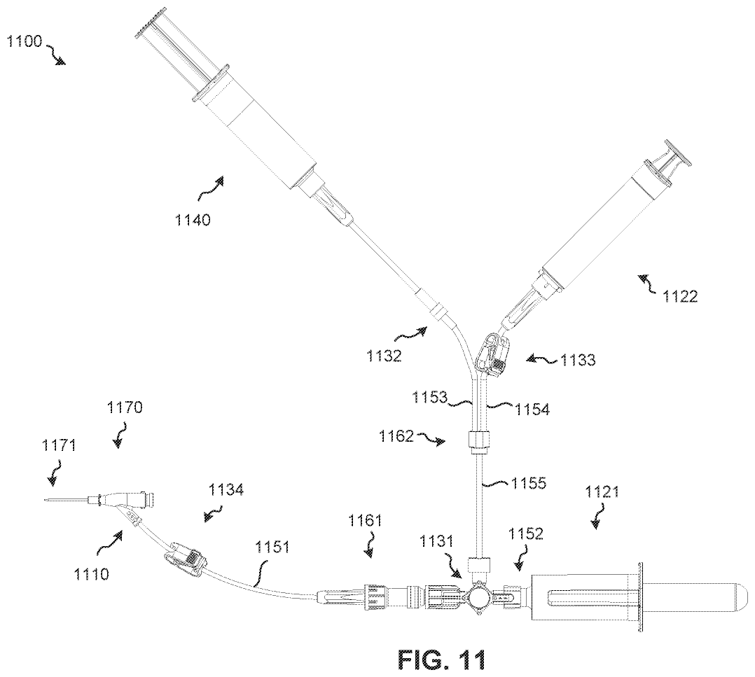

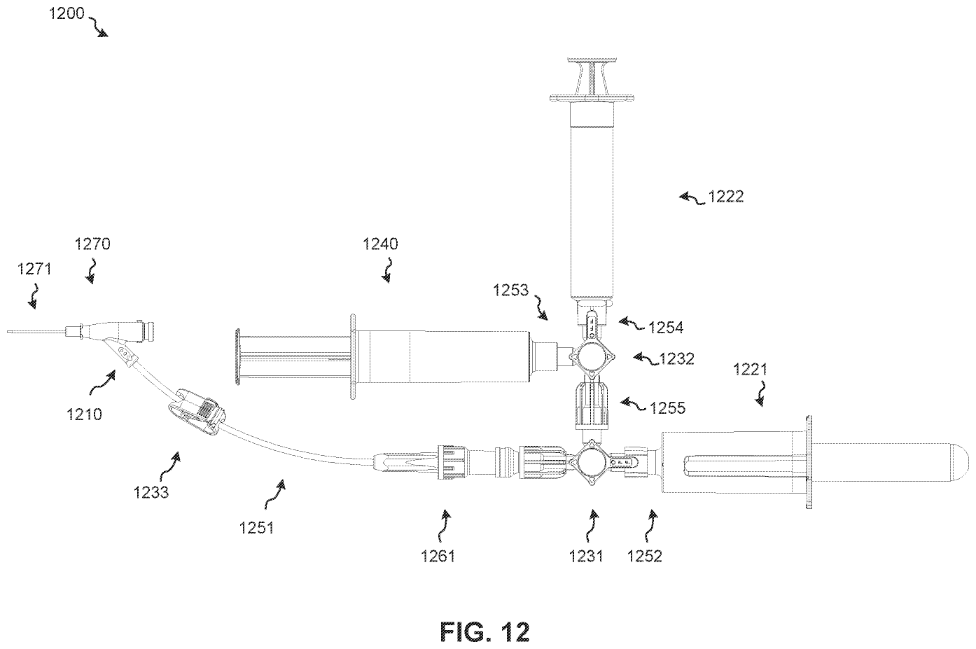

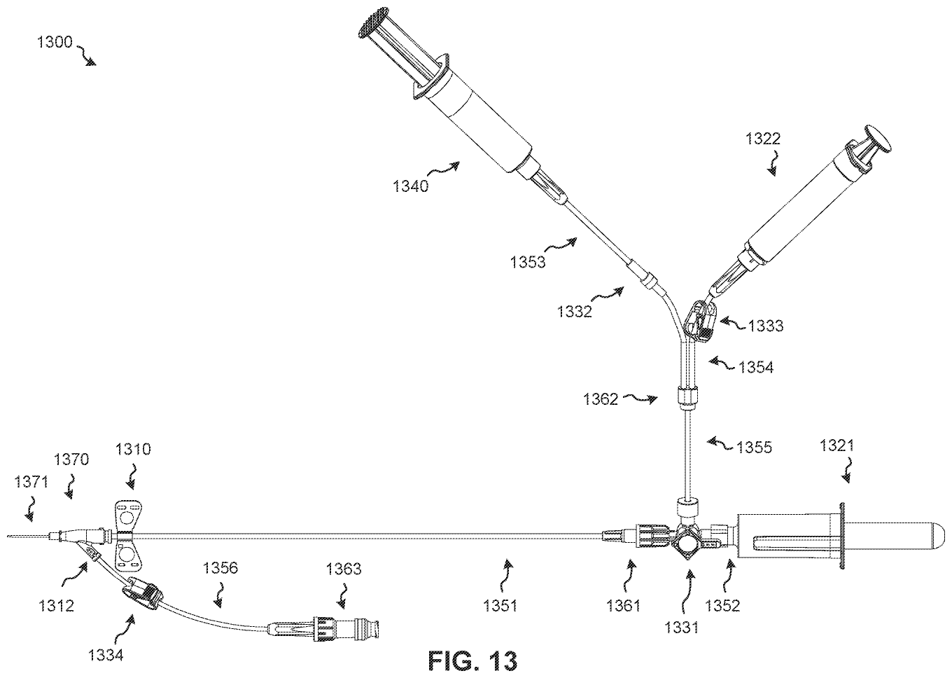

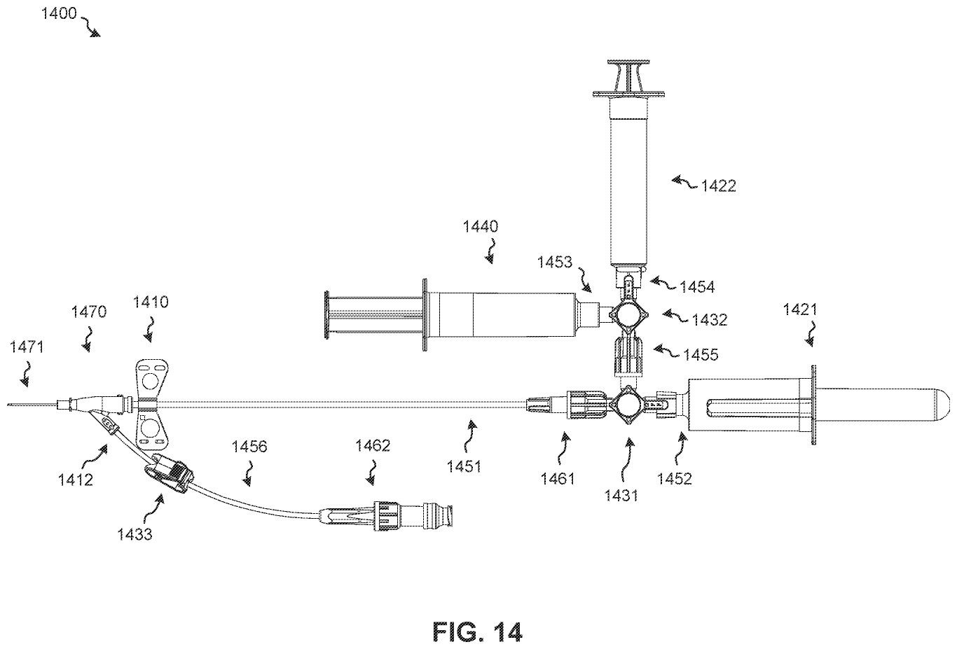

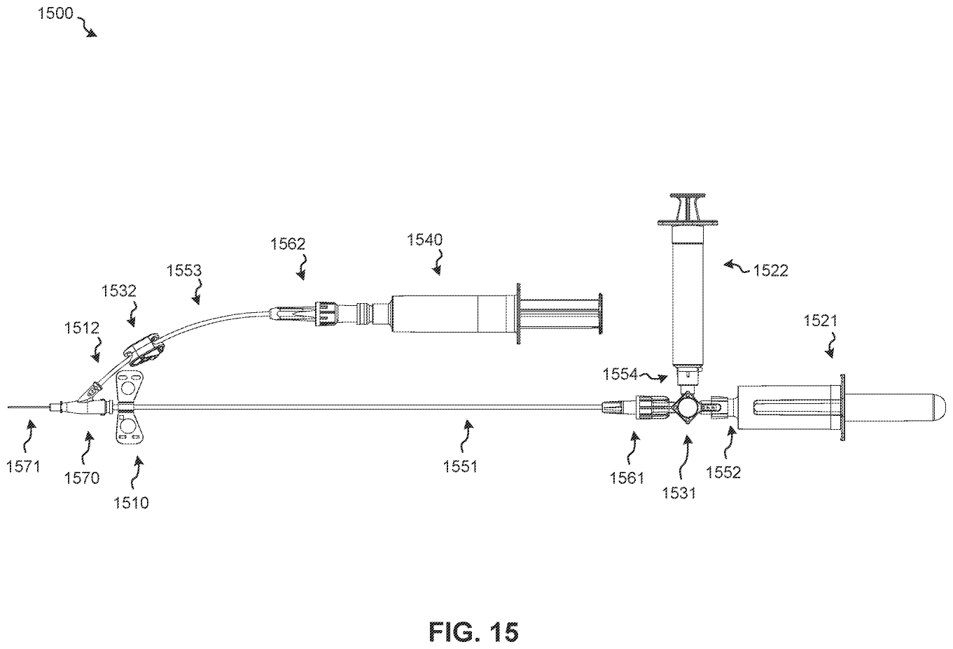

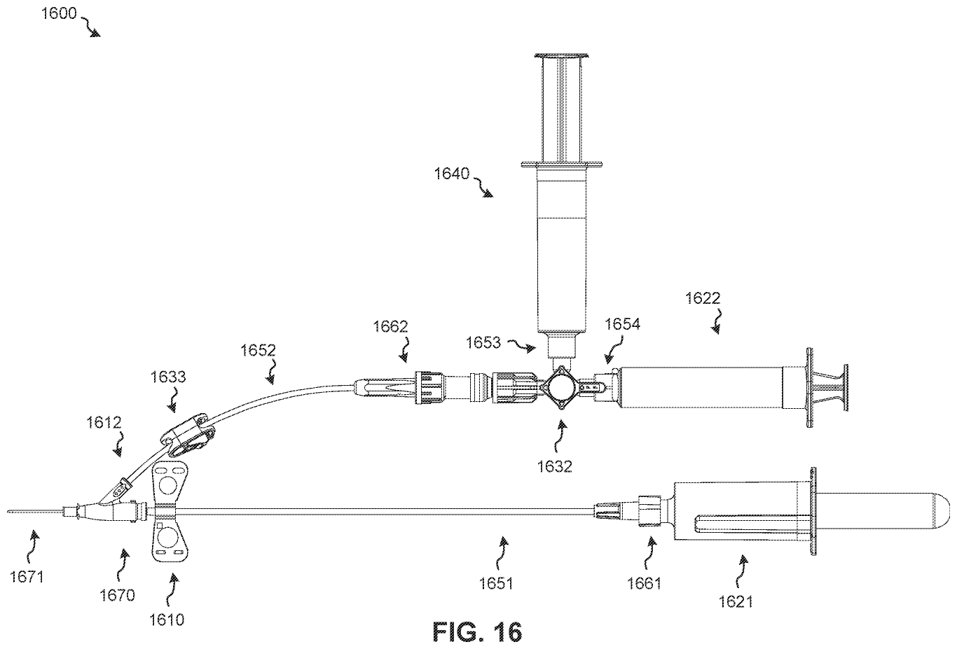

FIGS. 10-16 illustrate various example blood draw collection sets, according to some embodiments. Specifically, FIG. 10 is a side view of an example blood collection set 1000; FIG. 11 is a side view of an example blood collection set 1100; FIG. 12 is a side view of an example blood collection set 1200; FIG. 13 is a side view of an example blood collection set 1300; FIG. 14 is a side view of an example blood collection set 1400; FIG. 15 is a side view of an example blood collection set 1500; and FIG. 16 is a side view of an example blood collection set 1600.

The example blood collection set 1000 shown in FIG. 10 may generally include a first catheter adapter connector 1010, a first fluid conduit 1051, a second fluid conduit 1052, a third fluid conduit 1053, a first connector 1061, a first valve 1031, a second valve 1032, a first blood draw device 1021, and a flush device 1040.

The first catheter adapter connector 1010 may be configured to engage with a catheter adapter (not shown) in order to place the first catheter adapter connector 1010 in fluid communication with the catheter adapter. The catheter adapter may also include a catheter, which may be placed within a blood vessel of a patient (not shown). The first catheter adapter connector 1010, as well as any other catheter adapter connector disclosed herein, may include at least one of a needle, a wing needle set, a needleless connector (e.g., a Q-SYTE™ connector, a MAX ZERO™ connector, a SMARTSITE™ connector, etc.), a luer lock connector, a lure slip connector, a luer taper connector, an extension set, and/or an extension tube.

The first blood draw device 1021 may be placed in selective fluid communication with the first catheter adapter connector 1010 via the first fluid conduit 1051 and the second fluid conduit 1052, which may be coupled together via the first connector 1061. The first blood draw device 1021, as well as any other blood draw device disclosed herein, may be configured to draw blood from the blood vessel of the patient and may include at least one of a syringe and a VACUTAINER®.

The first valve 1031 may be configured to selectively permit fluid communication between the first blood draw device 1021 and the first catheter adapter connector 1010. The first valve 1031, as well as any other valve disclosed herein, may include at least one of a stopcock valve, a two-way stopcock valve, a three-way stopcock valve, a check valve, a slide clamp, and a pinch clamp. Once the first valve 1031 has been moved to an open position, the clinician may draw blood into the first blood draw device 1021. The first valve 1031 may then be moved to a closed position for subsequent steps during a blood draw procedure, as desired.

The flush device 1040 may be configured to flush the catheter adapter with fluid ejected from the flush device 1040 after blood has been drawn from the patient. In some embodiments, the flush device 1040 may include a syringe filled with a saline solution, such as a PosiFlush™. The flush device 1040 may be placed in selective fluid communication with the first catheter adapter connector 1010 via the first fluid conduit 1051 and the third fluid conduit 1053, which may be coupled together via the first connector 1061.

The second valve 1032 may be configured to selectively permit fluid communication between the flush device 1040 and the first catheter adapter connector 1010. Once the second valve 1032 has been moved to an open position, the clinician may flush the catheter adapter with fluid ejected from the flush device 1040. The second valve 1032 may then be moved to a closed position for subsequent steps during a blood draw procedure, as desired.

The example blood collection set 1100 shown in FIG. 11 may generally include a first catheter adapter connector 1110, a first fluid conduit 1151, a second fluid conduit 1152, a third fluid conduit 1153, a fourth fluid conduit 1154, a fifth fluid conduit 1155, a first connector 1161, a second connector 1162, a first valve 1131, a second valve 1132, a third valve 1133, a fourth valve 1134, a first blood draw device 1121, a second blood draw device 1122, and a flush device 1140.

The first catheter adapter connector 1110 may be configured to engage with a catheter adapter 1170 in order to place the first catheter adapter connector 1110 in fluid communication with the catheter adapter 1170. The catheter adapter 1170 may also include a catheter 1171, which may be placed within a blood vessel of a patient (not shown).

The first blood draw device 1121 may be placed in selective fluid communication with the first catheter adapter connector 1110 via the first fluid conduit 1151 and the second fluid conduit 1152, which may be fluidly coupled via the first connector 1161.

The first valve 1131 and/or the fourth valve 1134 may be configured to selectively permit fluid communication between the first blood draw device 1121 and the first catheter adapter connector 1110. Once the first valve 1131 and/or the fourth valve 1134 have been moved to open positions, the clinician may draw blood into the first blood draw device 1121. The first valve 1131 and/or the fourth valve 1134 may then be moved to closed positions for subsequent steps during a blood draw procedure, as desired.

The second blood draw device 1122 may be placed in selective fluid communication with the first catheter adapter connector 1110 via the first fluid conduit 1151, the fourth fluid conduit 1154 and the fifth fluid conduit 1155, which may be fluidly coupled via the first connector 1161 and/or the second connector 1162.

The first valve 1131, the third valve 1133, and/or the fourth valve 1134 may each be configured to selectively permit fluid communication between the second blood draw device 1122 and the first catheter adapter connector 1110. Once the first valve 1131, the third valve 1133, and/or the fourth valve 1134 have each been moved to open positions, the clinician may draw blood into the second blood draw device 1122. The first valve 1131, the third valve 1133, and/or the fourth valve 1134 may then be moved to closed positions for subsequent steps during a blood draw procedure, as desired. In some embodiments, blood may be drawn into the second blood draw device 1122 in order to remove blood from the patient's vein containing one or more drugs (e.g., from a prior infusion) before a clean blood sample may then be drawn into the first blood draw device 1121.

The flush device 1140 may be configured to flush the catheter adapter 1170 with fluid ejected from the flush device 1140 after blood has been drawn from the patient. The flush device 1140 may be placed in selective fluid communication with the first catheter adapter connector 1110 via the first fluid conduit 1151, the third fluid conduit 1153, and the fifth fluid conduit 1155, which may be fluidly coupled via the first connector 1161 and/or the second connector 1162.

The first valve 1131, the second valve 1132, and/or the fourth valve 1134 may each be configured to selectively permit fluid communication between the flush device 1140 and the first catheter adapter connector 1110. Once the first valve 1131, the second valve 1132, and/or the fourth valve 1134 have been moved to open positions, the clinician may flush the catheter adapter 1170 with fluid ejected from the flush device 1140. The first valve 1131, the second valve 1132, and/or the fourth valve 1134 may then be moved to closed positions for subsequent steps during a blood draw procedure, as desired.

The example blood collection set 1200 shown in FIG. 12 may generally include a first catheter adapter connector 1210, a first fluid conduit 1251, a second fluid conduit 1252, a third fluid conduit 1253, a fourth fluid conduit 1254, a fifth fluid conduit 1255, a first connector 1261, a first valve 1231, a second valve 1232, a third valve 1233, a first blood draw device 1221, a second blood draw device 1222, and a flush device 1240.

The first catheter adapter connector 1210 may be configured to engage with a catheter adapter 1270 in order to place the first catheter adapter connector 1210 in fluid communication with the catheter adapter 1270. The catheter adapter 1270 may also include a catheter 1271, which may be placed within a blood vessel of a patient (not shown).

The first blood draw device 1221 may be placed in selective fluid communication with the first catheter adapter connector 1210 via the first fluid conduit 1251 and the second fluid conduit 1252, which may be fluidly coupled via the first connector 1261.

The first valve 1231 and/or the third valve 1233 may be configured to selectively permit fluid communication between the first blood draw device 1221 and the first catheter adapter connector 1210. Once the first valve 1231 and/or third valve 1233 have been moved to open positions, the clinician may draw blood into the first blood draw device 1221. The first valve 1231 and/or third valve 1233 may then be moved to closed positions for subsequent steps during a blood draw procedure, as desired.

The second blood draw device 1222 may be placed in selective fluid communication with the first catheter adapter connector 1210 via the first fluid conduit 1251, the fourth fluid conduit 1254 and the fifth fluid conduit 1255, which may be fluidly coupled via the first connector 1261.

The first valve 1231, the second valve 1232, and/or the third valve 1233 may each be configured to selectively permit fluid communication between the second blood draw device 1222 and the first catheter adapter connector 1210. Once the first valve 1231, the second valve 1232, and/or the third valve 1233 have each been moved to open positions for the second blood draw device 1222, the clinician may draw blood into the second blood draw device 1222. The first valve 1231, the second valve 1232, and/or the third valve 1233 may then be moved to closed positions for the second blood draw device 1222 during subsequent steps of a blood draw procedure, as desired. In some embodiments, blood may be drawn into the second blood draw device 1222 in order to remove blood from the patient's vein containing one or more drugs (e.g., from a prior infusion) before a clean blood sample may then be drawn into the first blood draw device 1221.

The flush device 1240 may be configured to flush the catheter adapter 1270 with fluid ejected from the flush device 1240 after blood has been drawn from the patient. The flush device 1240 may be placed in selective fluid communication with the first catheter adapter connector 1210 via the first fluid conduit 1251, the third fluid conduit 1253, and the fifth fluid conduit 1255, which may be fluidly coupled via the first connector 1261.

The first valve 1231, the second valve 1232, and/or the third valve 1233 may each be configured to selectively permit fluid communication between the flush device 1240 and the first catheter adapter connector 1210. Once the first valve 1231, the second valve 1232, and/or the third valve 1233 have each been moved to open positions for the flush device 1240, the clinician may flush the catheter adapter 1270 with fluid ejected from the flush device 1240. The first valve 1231, the second valve 1232, and/or third valve 1233 may then be moved to closed positions for the flush device 1240 during subsequent steps of a blood draw procedure, as desired.

The example blood collection set 1300 shown in FIG. 13 may generally include a first catheter adapter connector 1310, a second catheter adapter connector 1312, a first fluid conduit 1351, a second fluid conduit 1352, a third fluid conduit 1353, a fourth fluid conduit 1354, a fifth fluid conduit 1355, a sixth fluid conduit 1356, a first connector 1361, a second connector 1362, a third connector 1363, a first valve 1331, a second valve 1332, a third valve 1333, a fourth valve 1334, a first blood draw device 1321, a second blood draw device 1322, and a flush device 1340.

The first catheter adapter connector 1310 may be configured to engage with a catheter adapter 1370 in order to place the first catheter adapter connector 1310 in fluid communication with the catheter adapter 1370. The catheter adapter 1370 may also include a catheter 1371, which may be placed within a blood vessel of a patient (not shown).

The first blood draw device 1321 may be placed in selective fluid communication with the first catheter adapter connector 1310 via the first fluid conduit 1351 and the second fluid conduit 1352, which may be fluidly coupled via the first connector 1361.

The first valve 1331 may be configured to selectively permit fluid communication between the first blood draw device 1321 and the first catheter adapter connector 1310. Once the first valve 1331 has been moved to an open position, the clinician may draw blood into the first blood draw device 1321. The first valve 1331 may then be moved to a closed position for subsequent steps during a blood draw procedure, as desired.

The second blood draw device 1322 may be placed in selective fluid communication with the first catheter adapter connector 1310 via the first fluid conduit 1351, the fourth fluid conduit 1354, and the fifth fluid conduit 1355, which may be fluidly coupled via the first connector 1361 and/or the second connector 1362.

The first valve 1331 and/or the third valve 1333 may each be configured to selectively permit fluid communication between the second blood draw device 1322 and the first catheter adapter connector 1310. Once the first valve 1331 and/or the third valve 1333 have each been moved to open positions, the clinician may draw blood into the second blood draw device 1322. The first valve 1331 and/or the third valve 1333 may then be moved to closed positions for subsequent steps during a blood draw procedure, as desired. In some embodiments, blood may be drawn into the second blood draw device 1322 in order to remove blood from the patient's vein containing one or more drugs (e.g., from a prior infusion) before a clean blood sample may then be drawn into the first blood draw device 1321.

The flush device 1340 may be configured to flush the catheter adapter 1370 with fluid ejected from the flush device 1340 after blood has been drawn from the patient. The flush device 1340 may be placed in selective fluid communication with the first catheter adapter connector 1310 via the first fluid conduit 1351, the third fluid conduit 1353, and the fifth fluid conduit 1355, which may be fluidly coupled via the first connector 1361 and/or the second connector 1362.

The first valve 1331 and/or the second valve 1332 may each be configured to selectively permit fluid communication between the flush device 1340 and the first catheter adapter connector 1310. Once the first valve 1331 and/or the second valve 1332 have each been moved to open positions, the clinician may flush the catheter adapter 1370 with fluid ejected from the flush device 1340. The first valve 1331 and/or the second valve 1332 may then be moved to closed positions for subsequent steps during a blood draw procedure, as desired.

The example blood collection set 1400 shown in FIG. 14 may generally include a first catheter adapter connector 1410, a second catheter adapter connector 1412, a first fluid conduit 1451, a second fluid conduit 1452, a third fluid conduit 1453, a fourth fluid conduit 1454, a fifth fluid conduit 1455, a sixth fluid conduit 1456, a first connector 1461, a second connector 1462, a first valve 1431, a second valve 1432, a third valve 1433, a first blood draw device 1421, a second blood draw device 1422, and a flush device 1440.

The first catheter adapter connector 1410 may be configured to engage with a catheter adapter 1470 in order to place the first catheter adapter connector 1410 in fluid communication with the catheter adapter 1470. The catheter adapter 1470 may also include a catheter 1471, which may be placed within a blood vessel of a patient (not shown).

The first blood draw device 1421 may be placed in selective fluid communication with the first catheter adapter connector 1410 via the first fluid conduit 1451 and the second fluid conduit 1452, which may be fluidly coupled via the first connector 1461.

The first valve 1431 may be configured to selectively permit fluid communication between the first blood draw device 1421 and the first catheter adapter connector 1410. Once the first valve 1431 has been moved to an open position, the clinician may draw blood into the first blood draw device 1421. The first valve 1431 may then be moved to a closed position for subsequent steps during a blood draw procedure, as desired.

The second blood draw device 1422 may be placed in selective fluid communication with the first catheter adapter connector 1410 via the first fluid conduit 1451, the fourth fluid conduit 1454, and the fifth fluid conduit 1455, which may be fluidly coupled via the first connector 1461.

The first valve 1431 and/or the second valve 1432 may each be configured to selectively permit fluid communication between the second blood draw device 1422 and the first catheter adapter connector 1410. Once the first valve 1431 and/or the second valve 1432 have each been moved to open positions for the second blood draw device 1422, the clinician may draw blood into the second blood draw device 1422. The first valve 1431 and/or the second valve 1432 may then be moved to closed positions for the second blood draw device 1422 during subsequent steps of a blood draw procedure, as desired. In some embodiments, blood may be drawn into the second blood draw device 1422 in order to remove blood from the patient's vein containing one or more drugs (e.g., from a prior infusion) before a clean blood sample may then be drawn into the first blood draw device 1421.

The flush device 1440 may be configured to flush the catheter adapter 1470 with fluid ejected from the flush device 1440 after blood has been drawn from the patient. The flush device 1440 may be placed in selective fluid communication with the first catheter adapter connector 1410 via the first fluid conduit 1451, the third fluid conduit 1453, and the fifth fluid conduit 1455, which may be fluidly coupled via the first connector 1461.

The first valve 1431 and/or the second valve 1432 may each be configured to selectively permit fluid communication between the flush device 1440 and the first catheter adapter connector 1410. Once the first valve 1431 and/or the second valve 1432 have each been moved to open positions for the flush device 1440, the clinician may flush the catheter adapter 1470 with fluid ejected from the flush device 1440. The first valve 1431 and/or the second valve 1432 may then be moved to closed positions for the flush device 1440 during subsequent steps of a blood draw procedure, as desired.

The example blood collection set 1500 shown in FIG. 15 may generally include a first catheter adapter connector 1510, a second catheter adapter connector 1512, a first fluid conduit 1551, a second fluid conduit 1552, a third fluid conduit 1553, a fourth fluid conduit 1554, a first connector 1561, a second connector 1562, a first valve 1531, a second valve 1532, a first blood draw device 1521, a second blood draw device 1522, and a flush device 1540.

The first catheter adapter connector 1510 may be configured to engage with a catheter adapter 1570 in order to place the first catheter adapter connector 1510 in fluid communication with the catheter adapter 1570. The catheter adapter 1570 may also include a catheter 1571, which may be placed within a blood vessel of a patient (not shown).

The first blood draw device 1521 may be placed in selective fluid communication with the first catheter adapter connector 1510 via the first fluid conduit 1551 and the second fluid conduit 1552, which may be fluidly coupled via the first connector 1561.

The first valve 1531 may be configured to selectively permit fluid communication between the first blood draw device 1521 and the first catheter adapter connector 1510. Once the first valve 1531 has been moved to an open position for the first blood draw device 1521, the clinician may draw blood into the first blood draw device 1521. The first valve 1531 may then be moved to a closed position for subsequent steps during a blood draw procedure, as desired.

The second blood draw device 1522 may be placed in selective fluid communication with the first catheter adapter connector 1510 via the first fluid conduit 1551 and the fourth fluid conduit 1554, which may be fluidly coupled via the first connector 1561.

The first valve 1531 may be configured to selectively permit fluid communication between the second blood draw device 1522 and the first catheter adapter connector 1510. Once the first valve 1531 has been moved to an open position for the second blood draw device 1522, the clinician may draw blood into the second blood draw device 1522. The first valve 1531 may then be moved to a closed position for the second blood draw device 1522 during subsequent steps of a blood draw procedure, as desired. In some embodiments, blood may be drawn into the second blood draw device 1522 in order to remove blood from the patient's vein containing one or more drugs (e.g., from a prior infusion) before a clean blood sample may then be drawn into the first blood draw device 1521.

The flush device 1540 may be configured to flush the catheter adapter 1570 with fluid ejected from the flush device 1540 after blood has been drawn from the patient. The flush device 1540 may be placed in selective fluid communication with the second catheter adapter connector 1512 via the third fluid conduit 1553, which may be fluidly coupled to the flush device 1540 via the second connector 1562.

The second valve 1532 may each be configured to selectively permit fluid communication between the flush device 1540 and the second catheter adapter connector 1512. Once the second valve 1532 has been moved to an open position for the flush device 1540, the clinician may flush the catheter adapter 1570 with fluid ejected from the flush device 1540. The second valve 1532 may then be moved to a closed position for the flush device 1540 during subsequent steps of a blood draw procedure, as desired.

The example blood collection set 1600 shown in FIG. 16 may generally include a first catheter adapter connector 1610, a second catheter adapter connector 1612, a first fluid conduit 1651, a second fluid conduit 1652, a third fluid conduit 1653, a fourth fluid conduit 1654, a first connector 1661, a second connector 1662, a first valve (not shown), a second valve 1632, a third valve 1633, a first blood draw device 1621, a second blood draw device 1622, and a flush device 1640.

The first catheter adapter connector 1610 may be configured to engage with a catheter adapter 1670 in order to place the first catheter adapter connector 1610 in fluid communication with the catheter adapter 1670. The catheter adapter 1670 may also include a catheter 1671, which may be placed within a blood vessel of a patient (not shown).

The first blood draw device 1621 may be placed in selective fluid communication with the first catheter adapter connector 1610 via the first fluid conduit 1651, which may be fluidly coupled with the first blood draw device 1621 via the first connector 1661.

A first valve (not shown) may be coupled to the first fluid conduit 1651 and may be configured to selectively permit fluid communication between the first blood draw device 1621 and the first catheter adapter connector 1610. Once the first valve has been moved to an open position for the first blood draw device 1621, the clinician may draw blood into the first blood draw device 1621. The first valve may then be moved to a closed position for subsequent steps during a blood draw procedure, as desired.

The second blood draw device 1622 may be placed in selective fluid communication with the second catheter adapter connector 1612 via the second fluid conduit 1652 and the fourth fluid conduit 1654, which may be fluidly coupled via the second connector 1662.

The second valve 1632 and/or the third valve 1633 may each be configured to selectively permit fluid communication between the second blood draw device 1622 and the second catheter adapter connector 1612. Once the second valve 1632 and/or the third valve 1633 have each been moved to open positions for the second blood draw device 1622, the clinician may draw blood into the second blood draw device 1622. The second valve 1632 and/or the third valve 1633 may then be moved to closed positions for the second blood draw device 1622 during subsequent steps of a blood draw procedure, as desired. In some embodiments, blood may be drawn into the second blood draw device 1622 in order to remove blood from the patient's vein containing one or more drugs (e.g., from a prior infusion) before a clean blood sample may then be drawn into the first blood draw device 1621.

The flush device 1640 may be configured to flush the catheter adapter 1670 with fluid ejected from the flush device 1640 after blood has been drawn from the patient. The flush device 1640 may be placed in selective fluid communication with the second catheter adapter connector 1612 via the second fluid conduit 1652 and the third fluid conduit 1653, which may be fluidly coupled via the second connector 1662.

The second valve 1632 and/or the third valve 1633 may each be configured to selectively permit fluid communication between the flush device 1640 and the second catheter adapter connector 1612. Once the second valve 1632 and/or the third valve 1633 have been moved to open positions for the flush device 1640, the clinician may flush the catheter adapter 1670 with fluid ejected from the flush device 1640. The second valve 1632 and/or the third valve 1633 may then be moved to closed positions for the flush device 1640 during subsequent steps of a blood draw procedure, as desired.

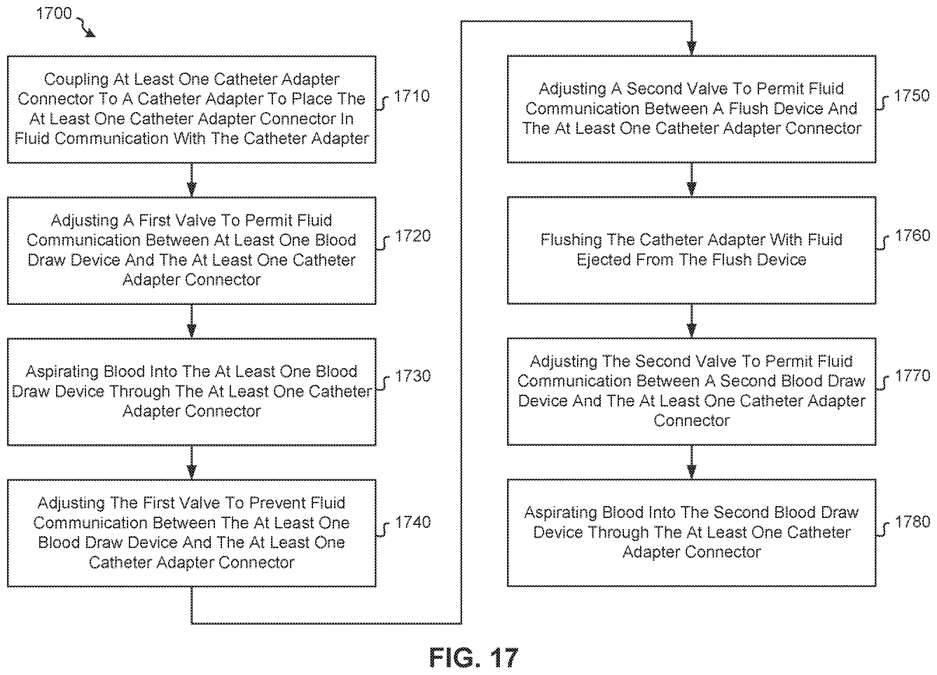

FIG. 17 is a flowchart of a method 1700 for drawing blood from a patient via a blood draw system, according to some embodiments. In general, the method 1700 may include the use of any blood collection set or any portion of any blood collection set disclosed herein.

The method 1700 may begin with a step 1710 in which at least one catheter adapter connector may be coupled to a catheter adapter in order to place the at least one catheter adapter connector in fluid communication with the catheter adapter. The catheter adapter may also include a catheter, which may be placed within a blood vessel of a patient.

Once the catheter adapter connector has been coupled to the catheter adapter, the method 1700 may proceed to a step 1720 in which a first valve may be adjusted to permit fluid communication between at least one blood draw device and the at least one catheter adapter connector. The at least one blood draw device may be configured to draw blood from a blood vessel of a patient via the catheter, the catheter adapter, the catheter adapter connector, and one or more fluid conduits.

Once the first valve has been adjusted to permit fluid communication between the at least one blood draw device and the at least one catheter adapter connector, the method 1700 may proceed to a step 1730 in which blood may be aspirated into the at least one blood draw device through the at least one catheter adapter connector.

Once the blood has been aspirated into the at least one blood draw device through the at least one catheter adapter connector, the method 1700 may proceed to a step 1740 in which the first valve may be adjusted to prevent fluid communication between the at least one blood draw device and the at least one catheter adapter connector.

Once the first valve has been adjusted to prevent fluid communication between the at least one blood draw device and the at least one catheter adapter connector, the method 1700 may proceed to a step 1750 in which a second valve may be adjusted to permit fluid communication between a flush device and the at least one catheter adapter connector.

Once the second valve has been adjusted to permit fluid communication between a flush device and the at least one catheter adapter connector, the method 1700 may proceed to a step 1760 in which the catheter adapter may be flushed with fluid ejected from the flush device.

Alternatively, or in addition thereto, the method 1700 may also include any one or more of the following steps, which may be performed in any order: (1) a step 1770 in which the second valve may be adjusted to prevent fluid communication between the flush device and the at least one catheter adapter connector and to permit fluid communication between a second blood draw device and the at least one catheter adapter connector; and (2) a step 1780 in which blood may be aspirated into the second blood draw device through the at least one catheter adapter connector.

Any methods disclosed herein include one or more steps or actions for performing the described method. One or more of the method steps and/or actions may be omitted from any of the methods disclosed herein. Moreover, any of the method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment. It is to be understood that any of the embodiments of the present disclosure, or any portion(s) of any of the embodiments of the present disclosure, may be combined together in any number of different ways.

Similarly, it should be appreciated that in the above description of embodiments, various features are sometimes grouped together in a single embodiment, Figure, or description thereof for the purpose of streamlining the disclosure. This disclosure format, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Description Of Embodiments are hereby expressly incorporated into this Description Of Embodiments, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

Standard medical directions, planes of reference, and descriptive terminology are employed in this specification. For example, anterior means toward the front of the body. Posterior means toward the back of the body. Superior means toward the head. Inferior means toward the feet. Medial means toward the midline of the body. Lateral means away from the midline of the body. Axial means toward a central axis of the body. Abaxial means away from a central axis of the body. Ipsilateral means on the same side of the body. Contralateral means on the opposite side of the body. A sagittal plane divides a body into right and left portions. A midsagittal plane divides the body into bilaterally symmetric right and left halves. A coronal plane divides a body into anterior and posterior portions. A transverse plane divides a body into superior and inferior portions. These descriptive terms may be applied to an animate or inanimate body.

The phrases “connected to,” “coupled to,” “engaged with,” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature is able to pass into the other feature.

As defined herein, “substantially equal to” means “equal to,” or within about a + or −10% relative variance from one another.

The word “example” is used herein to mean “serving as an example, instance, or illustration.” Any embodiment described herein as “example” is not necessarily to be construed as preferred or advantageous over other embodiments. While the various aspects of the embodiments are presented in the Figures, the Figures are not necessarily drawn to scale unless specifically indicated.

While specific embodiments and applications of the present disclosure have been illustrated and described, it is to be understood that the scope of the appended claims is not limited to the precise configuration and components disclosed herein. Various modifications, changes, and variations which will be apparent to those skilled in the art may be made in the arrangement, operation, and details of the apparatus and systems disclosed herein.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

1. A blood draw system comprising:
at least one catheter adapter connector configured to engage a catheter adapter and place the at least one catheter adapter connector in fluid communication with the catheter adapter;
at least one blood draw device in selective fluid communication with the at least one catheter adapter connector, the at least one blood draw device configured to draw blood from a blood vessel of a patient;
a first valve configured to selectively permit fluid communication between the at least one blood draw device and the at least one catheter adapter connector;
a flush device in selective fluid communication with the at least one catheter adapter connector, the flush device configured to flush the catheter adapter with fluid ejected from the flush device; and
a second valve configured to selectively permit fluid communication between the flush device and the at least one catheter adapter connector.

2. The blood draw system of claim 1, wherein the at least one catheter adapter connector comprises at least one of:
a needle;
a wing needle set;
a needleless connector;
a luer lock connector;
a lure slip connector;
a luer taper connector;
an extension set; and
an extension tube.

3. The blood draw system of claim 1, wherein the at least one blood draw device comprises at least one of:
a syringe; and
a vacutainer.

4. The blood draw system of claim 1, wherein the first valve and the second valve comprise at least one of:
a stopcock valve;
a two-way stopcock valve;
a three-way stopcock valve;
a check valve;
a slide clamp; and
a pinch clamp.

5. The blood draw system of claim 1, wherein the flush device comprises a syringe filled with a saline solution.

6. The blood draw system of claim 1, further comprising at least one fluid conduit coupled to the at least one catheter adapter connector, wherein the at least one fluid conduit is configured to place at least one of the flush device and the at least one blood draw device in fluid communication with the at least one catheter adapter connector.

7. The blood draw system of claim 1, wherein the at least one catheter adapter connector comprises a needle that is at least partially housed within a protective needle housing.

8. A protective needle housing system comprising:
a needle; and
a protective needle housing comprising:
a needle passageway configured to receive at least a portion of the needle therein; and
a needle block configured to move between a closed position and an open position;
wherein:
in the closed position, the needle block prevents the needle from advancing distally through the needle passageway; and
in the open position, an aperture formed in the needle block is placed in alignment with the needle passageway to allow the needle to advance distally through the needle passageway.

9. The protective needle housing system of claim 8, further comprising a resilient member configured to bias the needle block in the closed position.

10. The protective needle housing system of claim 9, wherein the resilient member comprises at least one of:
a spring clip; and
a helical spring.

11. The protective needle housing system of claim 8, wherein the needle block comprises a first engagement surface configured to engage with a second engagement surface of a catheter adapter, such that, as the second engagement surface of the catheter adapter engages the first engagement surface of the needle block, the needle block is moved from the closed position to the open position.

12. The protective needle housing system of claim 8, wherein the protective needle housing comprises an engagement feature configured to couple the protective needle housing to a catheter adapter.

13. The protective needle housing system of claim 8, wherein a distal end of the needle comprises:
a closed tip; and
an aspiration aperture formed in a sidewall of the needle proximate the closed tip.

14. The protective needle housing system of claim 8, wherein the needle comprises a bumper configured to prevent the needle from moving proximally out of the protective needle housing.

15. A method of drawing blood from a patient via a blood draw system, the method comprising:
coupling at least one catheter adapter connector to a catheter adapter in order to place the at least one catheter adapter connector in fluid communication with the catheter adapter;
adjusting a first valve to permit fluid communication between at least one blood draw device and the at least one catheter adapter connector, the at least one blood draw device configured to draw blood from a blood vessel of a patient;
aspirating blood into the at least one blood draw device through the at least one catheter adapter connector;
adjusting the first valve to prevent fluid communication between the at least one blood draw device and the at least one catheter adapter connector;
adjusting a second valve to permit fluid communication between a flush device and the at least one catheter adapter connector; and
flushing the catheter adapter with fluid ejected from the flush device.

16. The method of claim 15, further comprising:
adjusting the second valve to prevent fluid communication between the flush device and the at least one catheter adapter connector and to permit fluid communication between a second blood draw device and the at least one catheter adapter connector; and
aspirating blood into the second blood draw device through the at least one catheter adapter connector.

17. The method of claim 15, wherein the at least one catheter adapter connector comprises a first catheter adapter connector and a second catheter adapter connector.

18. The method of claim 15, wherein the at least one catheter adapter connector comprises at least one of:
a needle;
a wing needle set;
a needleless connector;
a luer lock connector;
a lure slip connector;
a luer taper connector;
an extension set; and
an extension tube.

19. The method of claim 18, wherein the at least one catheter adapter connector comprises a needle that is at least partially housed within a protective needle housing.

20. The method of claim 15, wherein the first valve and the second valve comprise at least one of:
a stopcock valve;
a two-way stopcock valve;
a three-way stopcock valve;
a check valve;
a slide clamp; and
a pinch clamp.
